(19) United States
(12) Patent Application Publication
(10) Pub. No.: US 2020/0268286 A1
(43) Pub. Date: Aug. 27, 2020

(54) MOVEMENT ASSESSMENT SYSTEM

(71) Applicant: AUSUSA Medical Innovations, Victoria (AU)

(72) Inventor: Ryan Butler, Victoria (AU)

(73) Assignee: AUSUSA Medical Innovations, Victoria (AU)

(21) Appl. No.: 16/484,316

(22) PCT Filed: Feb. 9, 2018
(86) PCT No.: PCT/AU2018/050098

§ 371 Date: Aug. 7, 2019

(30) Foreign Application Priority Data
Feb. 9, 2017 (AU) 2017900409

(51) Int. Cl. A61B 5/11; A61B 5/00; G08B 7/06; G08B 21/04

(52) U.S. Cl. CPC A61B 5/1115; A61B 5/7425; A61B 5/7405; A61B 5/7275; A61B 5/6889; A61B 2562/06; G08B 7/06; G08B 21/043; G08B 21/0492; A61B 2562/0247; A61B 2562/0233; A61B 5/6892

(57) ABSTRACT

A system for sensing and assessing movement, the system comprising a sensing arrangement for sensing movement in a space, the sensing arrangement comprising a plurality of sensors, at least some of which are adapted to sense movement on a surface and at least some of which are adapted to sense movement in a space; a receiving unit adapted to receive data from the sensing arrangement; a system for interpreting that data to provide an assessment of movement; an interface adapted to display that assessment of movement and provide a visual indicator of relative impact.

### TECHNICAL FIELD

This disclosure relates to a system for sensing and assessing movement in a space, limiting falls of a patient or other user and to a system for detecting the risk of a fall and providing a warning of that risk. The system is particularly described for use in a hospital system to prevent patient falls within a hospital room or from a bed, but is not limited to this use and can be available for use in other circumstances where detection and visual awareness of movement in multiple zones is necessary.

### BACKGROUND

Falls are a common cause of injury, particularly in a hospital system where individuals may be injured, bed-ridden, elderly or medicated to the extent that their ability to resist falls is prejudiced. Limiting further injury within a hospital is a critical to maintaining patient health. However it can be complicated for health practitioners to maintain constant watch of patients who may be unexpectedly in danger of falling.

### SUMMARY

Disclosed is a system for cognizance of movement or limiting falls, the system comprising a sensing arrangement for sensing movement, the sensing arrangement comprising a plurality of sensors, at least some of which are adapted to sense movement of a person on a surface such as a bed or chair or floor or other resting space, and at least some of which are adapted to sense movement of the patient in a space such as a room; a receiving unit adapted to receive data from the plurality of sensors making up the sensing arrangement; a system for interpreting that data to provide an assessment of risk; and an interface adapted to display that assessment of risk and provide an indicator of relative risk.

In some forms, the indicator is a visual indicator. In some form the visual indicator comprises a series of colours indicating relative risk.

In some forms the sensing arrangement comprises a combination of sensor types, including pressure sensors and infrared sensors.

The combination of sensors provides an accurate assessment of risk and awareness of movement, which is beneficially combined into a single interface. The interface can be easily and quickly analysed and is provided in a simple form such as, for example, a screen with colour or other visual indicators of risk levels. In some forms the interface may be able to be analysed from a distance or while moving without requiring a user to examine the screen in detail.

### BRIEF DESCRIPTION OF THE DRAWINGS

Embodiments will now be described by way of example only, with reference to the accompanying drawing in which

FIG. 1 is a schematic overview of a system of one embodiment of the disclosure.

### DETAILED DESCRIPTION

In the following detailed description, reference is made to accompanying drawing which forms a part of the detailed description. The illustrative embodiments described in the detailed description, depicted in the drawing and defined in the claims, are not intended to be limiting.

Disclosed is a system for providing an assessment of movement, the system comprising a sensing arrangement for sensing movement in a space, the sensing arrangement comprising a plurality of sensors, at least some of which are adapted to sense movement of a person on a surface such as a bed, chair, floor or other resting space, and at least some of which are adapted to sense movement in a space such as a room; a receiving unit adapted to receive data from the plurality of sensing arrangement; a system for interpreting that data to provide an assessment of movement; an interface adapted to display that assessment of movement and provide a visual indicator of movement.

In some form the system is specifically designed to provide an assessment of risk of patient falls.

In some forms, the visual indicator comprises a series of colours indicating relative risk of fall. In some forms red is used to indicate a high level of risk and green is used to indicate a low level of risk and at least one colour is used to indicate an intermediate level of risk. In some forms the series of colours are in a traffic light order, so green for low risk, yellow or orange for intermediate risk and red for high risk.

In some forms the interface further provides an aural indication of risk.

In some forms the system comprises a plurality of sensing arrangement for sensing movements in a plurality of spaces.

In some forms the system comprises a plurality of sensing arrangements for sensing the movement of a plurality of patients.

In some forms the interface is adapted to display a plurality of assessments of risk of a plurality of patients.

In some forms the sensors comprise at least one pressure sensor.

In some forms the sensors comprise at least one infrared sensor.

In some forms the sensors comprise at least one capacitance sensor alongside the pressure and infrared sensors.

In some forms movement detected by the sensing arrangement results in an assessment of increased risk. In some forms movement lasting longer than a pre-determined time and detected by the sensing arrangement results in an assessment of increased risk.

In some forms the sensing arrangement comprises a combination of sensor types, including pressure sensors and infrared sensors.

In some forms the sensing arrangement is adapted to be located in a room having a bed surface and at least one of the sensors senses movement of the patient on the bed.

This system allows for multiple spaces or multiple patients to be shown on a single screen, and for easy awareness by an individual staff of movements and in some forms of potential fall dangers or actual falls. It allows for ease of checking while combining multiple sensors. In some forms the combination of pressure and IR sensors or Pressure, IR and capacitance sensors provides a better picture of movement and associated risk. In some forms the use of coloured warnings in a traffic light format provides an easy and immediately translatable indication of movement and in some forms of fall risk. In some forms a person or the patient must move for greater than a certain designated period of time in order for the risk or warning to be triggered.

The system makes it easier to monitor multiple spaces or to monitor patients in multiple rooms and ensure highest priority is given to the spaces or people most needing it.

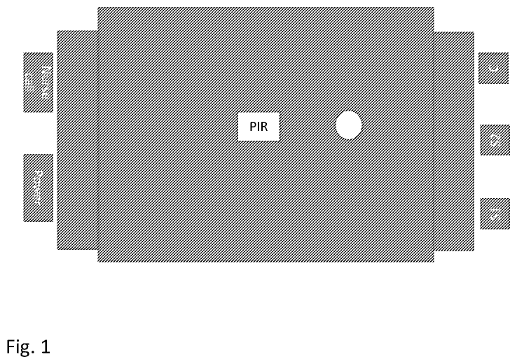

Referring now to FIG. 1, disclosed is a system for sensing and assessing movement and in some forms limiting patient falls.

The figure shows a general schematic of the system. The system is designed for use in a room such as a hospital room (not illustrated) and comprises a sensing arrangement (not illustrated) comprising a series of sensors working to sense movement of a patient to sense the risk of a fall and transmit that risk to a nurses' station or a device in order to reduce the risk of a fall.

For example, in some embodiments, the system comprises at least one pressure sensor in the form of a pressure mat on the bed, the pressure sensor being adapted to indicate if a person in or on the bed is present and/or if the person on the bed is moving. The pressure sensor senses weight on the sensor or movement of weight thereon.

The system also comprises at least one pressure sensor in the form of a pressure mat on the floor. The floor sensor is adapted to determine if a person is moving, lying or standing on the floor.

The sensing arrangement may also include at least one infrared sensor for determining if a person is moving in the room. The infrared sensor is a passive sensor that determines whether there is movement within the space and can be designed to fit a particular space within a ward or a particular room.

In some forms there may be further sensors in the sensing arrangement, including further pressure and infrared sensors. There may also be capacitance sensors for example in the form of a mat on the bed which sense a slight change in electrical field of a person.

In the form illustrated in FIG. 1, two pressure sensor inputs to the system are shown as S1 and S2. In some forms pressure sensors located on surfaces such as the bed and floor obtain data from pressure placed on those surfaces and input it into the system through inputs S1 and S2. In some forms the pressure sensors may include any combination of a bed mat pressure sensor, a floor mat pressure sensor, a chair mat pressure sensor. Pressure sensors can provide information about presence or absence of a person on a surface, about movement on a surface, about weight and positioning on a surface.

In the illustrated form, a third input labelled C allows input from a capacitance sensor. In some forms this may be a bed mat capacitance sensor or any other capacitance sensor.

A passive infrared sensor is in some forms also input into the system as indicated at PIR to provide input from the infrared sensor in the room.

In other forms further inputs can be incorporated into the system, such as further pressure sensors or any other sensor for sensing the position or movement of a patient.

The multiple sensors and multiple inputs allow for a careful determination of risk to a patient even if one sensor fails or is not sufficient to show movement.

In this form, further inputs such as a nurse call button NC and power input P are also incorporated on one side of the system. These allow for alternative inputs to indicate risk and priority level of the matter.

The data from the sensors and any further inputs may be analysed within the single user system or outside the system. The analysis of the data utilises information about the movement of a patient such as positioning, frequency, time period and extent of movement. This data is then analysed to determine whether the patient is moving and at risk or simply moving without risk or with limited risk. The data can be viewed for whether there is a potential danger to the patient or an imminent danger.

The determined data or the determined risk is output by wireless or wired technology to an easily viewed location such as, for example, the nurses' station or a device or monitor. A plurality of systems such as that generally indicated in FIG. 1 can be combined at the nurses' station, monitor, device or equivalent.

Data input into the system is analysed to determine whether a notification is necessary.

Table 1 shows one form of the notification logic of one embodiment of the disclosure.

| Inputs | Room | Bed | Floor | Capacitance | Logic |
|---|---|---|---|---|---|
| Green | PIR | P1 | P2 | C | |
| (output) | | | | | |
| | Off | Off | Off | Off | Patient on |
| | | | | | Bed/No |
| | | | | | Movement |
| Yellow | On (<3 s) | Off | Off | Off | Patient On |
| (output) | | | | | Bed/Some |
| | | | | | Movement |
| Red | X | X | On | x | Patient |
| (output) | | | | | off Bed |
| | On >3 Sec | x | x | x | Tolerance |
| | | | | | has broken |
| | x | On >3 Sec | x | x | Tolerance |
| | | | | | has broken |
| | x | x | x | On >3 Sec | Tolerance |
| | | | | | has broken |

For example, in the form shown in Table 1, the logic provides that if the passive infrared sensor, the two pressure sensors and the capacitance sensor of FIG. 1 are all off, indicating no movement, then the patient is on the bed and not moving and has a limited risk of a fall. As shown in the table, in the illustrated form when the risk of a fall is low, the output at the nurses' station or monitor shows as green output or low risk and an LED light or equivalent may also show green.

If the passive infrared sensor shows some movement as indicated in row 2 of the table, but the pressure and capacitance sensors remain off, then in this embodiment the logic of the system dictates that there is an intermediate risk of a fall. In this case the output is shown as yellow at the nurses' station, monitor or equivalent. In the alternative the output is linked to a yellow LED light or both the LED light and the output at the nurse's station are activated. In some forms the movement within the room must be for a time period of less than 3s to have an intermediate level of risk, though in alternative embodiments the movement may be for a period less than 1 or 2 second or less than 5 seconds or 10 seconds depending upon needs.

As shown in row 3 of the table, if there is pressure on the floor of any kind, regardless of movement or pressure on any other sensor, then the output will show a high level of risk as the patient is on the floor. This results in a red output at the nurses' station or equivalent, a red LED light, or both in order to provide a high indication of relative risk. Similarly as shown in row 4 if there is changing pressure on the bed for greater than three seconds or a shift in capacitance on the bed for greater than 3 seconds or movement in the room for greater than 3 seconds then the logic determines that the risk of a fall is high and the output is red to indicate that high risk. In some forms a further aural alarm sounds in these circumstances.

The notification logic of the system can be adjusted for different requirements depending upon the sensors, the room or the patient.

In some forms up to 8 or more inputs can be connected with the system.

In some forms the colour change output is in the form of tri-colour LEDs to indicate the sensing status. In some forms the output is in the form of a wireless connection with the nurse's station.

The software associated with a dedicated program on a given nurses' station or alternative computer is able to monitor the sensors, for example up to 16 sensors or more. The software displays patient and ward information along with the sensor status of up to 16 or more sensors in a grid shape. Thus it is easy for a health professional to monitor multiple beds and to determine priority of risk or that there is a high risk in one ward or with one patient. The level and location of risk can quickly be determined. This allows for better overall care of the patient.

In the claims which follow and in the preceding summary except where the context requires otherwise due to express language or necessary implication, the word “comprising” is used in the sense of “including”, that is, the features as above may be associated with further features in various embodiments.

In some not illustrated forms, the system may be utilized for sensing movement within a building or space. Multiple sensors provide an effective sensing system for movement. The multiple sensors are interlinked and engaged with an interface to provide an analysis of the movement in a simple to view screen form. In this form the potential impact indicated could be presence or movement of an unwanted person in a space. In other forms the impact could be slowing down or speeding up of movement in a space, numbers in a space or other wanted or unwanted impacts.

Variations and modifications may be made to the parts previously described without departing from the spirit or ambit of the disclosure.

What is claimed is:

1. A system for sensing and assessing movement, the system comprising:
a sensing arrangement for sensing movement in a space, the sensing arrangement comprising a plurality of sensors, at least some of which are adapted to sense movement on a surface and at least some of which are adapted to sense movement in a space;
a receiving unit adapted to receive data from the sensing arrangement;
a system for interpreting that data to provide an assessment of movement;
an interface adapted to display that assessment of movement and provide a visual indicator of relative impact.

2. The system of claim 1, wherein the visual indicator comprises a series of colours indicating relative impact.

3. The system of claim 2, wherein red is used to indicate a high level of impact and green is used to indicate a low level of impact and at least one colour is used to indicate an intermediate level of impact.

4. The system of claim 1, wherein the interface further provides an aural indication of impact.

5. The system of claim 1, wherein the system comprises a plurality of sensing arrangement for sensing movements in a plurality of spaces.

6. The system of claim 1, wherein interface is adapted to display a plurality of assessments of impact of a plurality of spaces.

7. The system of claim 1, wherein the sensors comprise at least one pressure sensor.

8. The system of claim 1, wherein the sensors comprise at least one infrared sensor.

9. The system of claim 1, wherein the sensors comprise at least one capacitance sensor.

10. The system of claim 1, wherein movement detected by the sensing arrangement results in an assessment of increased impact.

11. The system of claim 1, wherein movement lasting longer than a pre-determined time and detected by the sensing arrangement results in an assessment of increased impact.

12. The system of claim 1, wherein the sensing arrangement comprises a combination of sensor types, including pressure sensors and infrared sensors.

13. The system of claim 1, wherein the sensing arrangement is adapted to be located in a room having a bed surface and at least one of the sensors senses movement of the person on the bed.

14. The system of claim 1, wherein the system is designed for sensing patient movement.

15. The system of claim 1 any wherein the system is designed to provide an assessment of the risk of a patient fall.

16. The system of claim 14, wherein the system is designed to be located at a nurses station for easy access.

17. The system of claim 1 wherein the interface of the system is available on mobile devices.

18. A method of providing an indication of risk to a patient, the method comprising:
providing a plurality of sensors, at least some of which are adapted to sense movement on a surface and at least some of which are adapted to sense movement in a space;
receiving data from the sensors;
interpreting that data to provide an assessment of movement;
displaying that assessment of movement on an interface and providing an indicator of relative risk.

19. A system for providing an indication of risk to a patient, the system adapted to:
receive data from a plurality of sensors;
interpret that data to provide an assessment of movement;
display that assessment of movement on an interface and provide an indicator of relative risk.
